(19) United States
(12) Patent Application Publication
(10) Pub. No.: US 2010/0030330 A1
(43) Pub. Date: Feb. 4, 2010

(54) Device and method for mitral valve repair

(75) Inventors: Donald E. Bobo, Santa Ana, CA (US); Stanton Rowe, Newport beach, CA (US); Michael Popp, Orange, CA (US); Seung Yi, Aliso Viejo, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/221,450

(22) Filed: Aug. 1, 2008

(51) Int. Cl. A61F 2/24; A61B 19/00

(52) U.S. Cl. 623/2.36; 623/2.38; 128/898

(57) ABSTRACT

Devices and methods for reshaping a mitral valve annulus are provided. One device according to the invention is configured for deployment in the right atrium and is shaped to apply a force along the atrial septum. The device causes the atrial septum to deform and push the anterior leaflet of the mitral valve in a posterior direction for reducing mitral valve regurgitation. Another embodiment of a device is deployed in the left ventricular outflow tract at a location adjacent the aortic valve. The device may be expandable for urging the anterior leaflet toward the posterior leaflet. Another embodiment of the device includes a first anchor, a second anchor, and a bridge, with the bridge having sufficient length to reach from the coronary sinus to the right atrium and/or superior or inferior vena cava. In a further embodiment a device includes a middle anchor positioned on the bridge between the distal and proximal anchors.

### FIELD OF THE INVENTION

The present invention relates to medical devices and methods and, more particularly, to medical devices and methods for repairing a defective mitral valve in a human heart.

### BACKGROUND

Heart valve regurgitation, or leakage from the outflow to the inflow side of a heart valve, occurs when a heart valve fails to close properly. Regurgitation often occurs in the mitral valve, located between the left atrium and left ventricle, or in the tricuspid valve, located between the right atrium and right ventricle. Regurgitation through the mitral valve is often caused by changes in the geometric configurations of the left ventricle, papillary muscles, and/or mitral valve annulus. Similarly, regurgitation through the tricuspid valve is often caused by changes in the geometric configurations of the right ventricle, papillary muscles, and/or tricuspid valve annulus. These geometric alterations result in incomplete leaflet coaptation during ventricular systole, thereby producing regurgitation.

A variety of heart valve repair procedures have been proposed over the years for treating heart valve regurgitation. With the use of current surgical techniques, it has been found that a significant percentage of regurgitant heart valves can be repaired, depending on the surgeon's experience and the anatomic conditions present. Depending on various factors, such as the condition of a particular patient, heart valve repair can have advantages over heart valve replacement. These advantages include better preservation of cardiac function and reduced risk of anticoagulant-related hemorrhage, thromboembolism, and endocarditis.

In recent years, a variety of new minimally invasive procedures for repairing heart valves have been introduced. These minimally invasive procedures do not require opening the chest or the use of cardiopulmonary by-pass. At least one of these procedures involves introducing an implant into the coronary sinus for remodeling the mitral annulus. The coronary sinus is a blood vessel commencing at the coronary sinus ostium in the right atrium and passing through the atrioventricular groove in close proximity to the posterior, lateral, and medial aspects of the mitral annulus. Because the coronary sinus is positioned adjacent to the mitral valve annulus, an implant deployed within the coronary sinus may be used to apply a compressive force along a posterior portion of the mitral annulus for improving leaflet coaption.

Various implants configured for insertion into the coronary sinus for repairing mitral valves have been developed. For example, several patents to Solem et al., including U.S. Pat. No. 6,210,432, No. 6,997,951, No. 7,044,967, and No. 7,090,695, describe devices and methods for reducing mitral valve regurgitation via placement of a distal anchor within the great cardiac vein, a proximal anchor within or just adjacent the ostium of the coronary sinus, with the device including a cinching member connecting the two anchors and configured to draw the anchors together to cause a corresponding reshaping of the valve annulus.

Anchoring the device entirely within the coronary sinus and great cardiac vein is sufficient for treating many patients, depending on such factors as the positioning of the valve leaflets and corresponding line of coaptation with respect to the coronary sinus and other features, as well as the shape of the valve annulus and the amount of regurgitation pre-treatment. However, for other patients it may be desirable to anchor all or a portion of the device outside of the coronary sinus in order to achieve an annular reshaping that cannot be achieved by anchoring exclusively within the coronary sinus.

It is often the case with known implants that the proximal anchor is deployed directly adjacent to the P3 commissure location. Because the cinching action typically occurs distally of the proximal anchor, an implant thus deployed may have limited ability to reduce regurgitant area residing immediately adjacent the P3 commissure. Also, the length of existing devices, and thus the amount of cinching distance, is bounded by the length of the coronary sinus and great cardiac vein.

Although a variety of implants and delivery systems have been proposed for treating mitral valve regurgitation in a minimally invasive manner, many existing implants are limited in their ability to restructure the valve annulus. Known devices that extend from the coronary sinus ostium into the coronary sinus to the anterior interventricular vein (AIV) have significant ability to reshape the mitral valve, particularly where the patient's valve leaflets are oriented such that the line of leaflet coaptation with respect to the coronary sinus is acceptable. In some patients, however, the line of coaptation or other physical characteristics of the valve to be treated may require a different reshaping than can be achieved via an implant located essentially entirely within the coronary sinus.

Accordingly, a need exists for an improved implant sized to be anchored at least partially within a coronary sinus and with improved abilities to reshape a valve annulus for treating mitral valve regurgitation. It is desirable that such an implant include anchoring portions which are capable of securely engaging an interior wall of the coronary sinus as well as the right atrium, inferior vena cava, and/or superior vena cava. It is also desirable that such an implant be configured for percutaneous delivery and be relatively easy to manufacture. The present invention addresses these needs.

### SUMMARY OF THE INVENTION

Various embodiments of the present invention provide new devices and methods for treating heart valve regurgitation. The devices and methods are particularly well suited for treating mitral valve regurgitation in a minimally invasive manner.

In one embodiment, an implantable body is configured for deployment in the right atrium. The body is shaped to apply a lateral force along the atrial septum at a location adjacent to the mitral valve. The force causes the atrial septum to deform, thereby affecting the anatomy on the left side of the heart. More particularly, by pressing on the atrial septum, the anterior leaflet of the mitral valve is pushed toward the posterior leaflet. The amount of force can be selected such that the anterior leaflet is pushed a sufficient amount for closing the gap in the mitral valve and reducing or eliminating mitral valve regurgitation.

One device configured for this purpose generally comprises at least one anchor member for anchoring the device relative to the right atrium and a pusher member for engaging and pressing against the atrial septum. The anchor member may comprise an expandable stent configured for deployment in the superior vena cava. If desired, the anchor member may further comprise a second expandable stent configured for deployment in the inferior vena cava. The pusher member is coupled to the first and second anchors. The pusher member may comprise a bow-shaped member.

In another embodiment, a device is provided for placement in the right ventricle. In one aspect, the device comprises a ring or U-shaped member that changes shape for pushing against the ventricular septum.

In another embodiment, an expandable stent is configured for deployment in the left ventricular outflow tract. The expandable stent is adapted to exert a radial force for reshaping a mitral valve annulus, thereby moving an anterior leaflet of a mitral valve in a posterior direction. The device may be deployed at a location adjacent the aortic valve and, in some configurations, the device is deployed beneath the aortic valve. The stent may be configured with a protrusion to increase the force applied along the portion of the LVOT that is adjacent to the mitral valve. The stent may further comprise a valvular structure to provide a prosthetic valve configured for replacing an aortic valve, thereby providing a device configured to treat the aortic valve and mitral valve simultaneously.

In another aspect, a method of reducing mitral valve regurgitation comprises delivering an expandable body into the left ventricular outflow tract, wherein the expandable body is configured to urge the anterior leaflet of a mitral valve toward the posterior leaflet of a mitral valve, thereby improving leaflet coaption. In one variation, the expandable body may comprise a stent configured to be delivered into the left ventricular outflow tract in a minimally invasive manner. The stent may be delivered to a location in the left ventricular outflow tract just beneath the aortic valve.

In another embodiment, a tether or other tension member is provided for pulling the anterior leaflet toward the posterior leaflet. In one embodiment, the tether is located within the left ventricle. In another embodiment, the tether is located within the left atrium. The tether is configured to pull opposing regions of tissue into closer proximity for reshaping the mitral valve annulus.

In another aspect, a method for repairing a mitral valve involves providing a repair device having a deployment mechanism for independently applying first and second fastener elements to first and second regions of a mitral valve annulus. The repair device is used to grasp the first region of tissue with a vacuum force and then deploy a first fastener element into the first region of tissue. The first region of tissue is then disengaged from the repair device while leaving the first fastener element deployed therein. The repair device is then used to grasp the second region of tissue with a vacuum force and then deploy the second fastener element into the second region of tissue. The second region of tissue is then disengaged. The first and second fastener elements are then pulled together for reducing the distance between the first and second regions of tissue, thereby improving coaption of the mitral valve leaflets.

In one embodiment, an apparatus for treating a mitral valve includes: a distal anchor configured for deployment within a distal portion of the coronary sinus (e.g., great cardiac vein); a proximal anchor configured for deployment within the right atrium, inferior vena cava, and/or superior vena cava; and an elongate member connecting the distal and proximal anchors and configured to exert pressure to draw the distal anchor towards the proximal anchor. The device may also include an intermediate anchor secured to a mid-portion of the elongated member (i.e., between the distal and proximal anchors), with the intermediate anchor configured to be deployed within an intermediate area of the patient's body, e.g., within the ostium of the coronary sinus.

The elongate member may have a fixed length, or be configured to adjust or be adjusted from an elongated state to a shortened state before, during, or after delivery at least partially into a coronary sinus for reshaping a mitral annulus. One or more of the anchors (i.e., distal, proximal, and/or mid) may be secured in fixed position to specific points on the elongate member, and/or may be movably secured so as to be repositioned (e.g., slidingly) along the length of the elongate member.

The elongate member may be ioined to the anchors in various ways, including via ratchet-like and/or slidingly adjustable connection, flexible suture, loops, links, and/or hinge-like mechanisms. The implant may be formed from separate elements that are joined together by, for example, welding, crimping, bolting, or suturing. The implant may be made integrally from a single piece of material, such as wire, tube, ribbon, or plate.

Locating the proximal anchor outside of the coronary sinus can offer various advantages: The P3 commissure can be completely surrounded by the cinching mechanism, thereby improving the opportunities for reduction and/or elimination of any regurgitant orifice adjacent the P3 scallop; The securing ability of the anchors can be enhanced because the bridging element can be significantly longer and the bridges can be secured to areas having improved “holding” abilities; A one-size-fits-all device is possible because the right atrium, inferior vena cava, and superior vena cava exist entirely outside of the target area for cinching. Accurate placement of the proximal anchor is thus both easier to achieve and less critical to the procedure.

Additionally, methods for treating a mitral valve using an implant is provided, One method includes inserting the implant at least partially into the coronary sinus, anchoring the distal anchor in the coronary sinus, and anchoring the proximal anchor in the right atrium, superior vena cava, and/or inferior vena cava. The method may include, after deployment of the distal anchor but prior to deployment of the proximal anchor, pulling the proximal anchor in a proximal direction with respect to the distal anchor, then anchoring the proximal anchor in the right atrium and allowing the resorbable material to be resorbed, causing the bridge to shorten and thereby reshape a mitral annulus.

### BRIEF DESCRIPTION OF THE DRAWINGS

FIG. 1 is a first cross-sectional view of a typical four-chambered heart.

FIG. 2 is a cross-sectional view generally illustrating forces pushing against a septum for reshaping a mitral valve annulus.

FIG. 3 is a cross-sectional view generally illustrating one medical implant configured for applying a force along the atrial septum.

FIG. 3A is a schematic view illustrating the function of the implant of FIG. 3.

FIG. 3B illustrates the force acting on the anterior leaflet for urging the anterior leaflet toward the posterior leaflet.

FIG. 4 is a cross-sectional view generally illustrating another embodiment of a medical implant configured for applying a force along the ventricular septum.

FIG. 5 is a second cross-sectional view of a typical four-chambered heart.

FIG. 6 illustrates an expandable stent deployed in the left ventricular outflow tract for reshaping the mitral valve annulus.

FIG. 6A illustrates a cross-section of an expandable stent having a protrusion configured to apply a force along the anterior portion of the mitral valve annulus.

FIG. 7 illustrates yet another approach for treating a mitral valve wherein a tether extends across the left ventricle at a location beneath the mitral valve for improving mitral valve function.

FIG. 8 illustrates a tether attached to opposing regions of a mitral valve annulus at a location above the mitral valve for improving mitral valve function.

FIGS. 8A and 8B illustrate a method of attaching a tether to the mitral valve annulus.

FIGS. 8C through 8E illustrate various tether configurations for reshaping the mitral valve annulus.

FIG. 9 illustrates an alternative approach wherein one end of a tether is attached to chordae within the left ventricle.

FIG. 10 illustrates a prosthetic valve for replacing a native aortic valve and including a lower portion configured for reshaping the mitral valve annulus.

FIG. 11 illustrates a stent deployed in the right ventricular outflow tract for improving tricuspid valve function.

FIG. 12 is a top view of an implant according to an embodiment of the invention deployed in a right atrium and coronary sinus to reshape a mitral valve;

FIG. 13 is a side view of the implant of FIG. 12;

FIG. 14 is a top view of an implant according to a further embodiment of the invention deployed in a right atrium and coronary sinus to reshape a mitral valve;

FIG. 15 is a top view of an implant according to a further embodiment of the invention deployed in a right atrium and coronary sinus to reshape a mitral valve;

FIG. 16 is a top view of an implant according to a further embodiment of the invention deployed in a right atrium/inferior vena cava and coronary sinus to reshape a mitral valve;

FIG. 17 is a top view of an implant according to a further embodiment of the invention deployed in a right atrium/superior vena cava and coronary sinus to reshape a mitral valve;

FIG. 18 is a top view of an implant according to a further embodiment of the invention deployed in a right atrium/superior vena cava and coronary sinus to reshape a mitral valve;

FIG. 19A shows a guidewire advanced in the coronary sinus according to an embodiment of the present invention;

FIG. 19B shows a guide catheter and a dilator inserted over the guidewire to the coronary sinus according to an embodiment of the present invention;

FIG. 19C shows a guide catheter positioned over the guidewire at the coronary sinus according to an embodiment of the present invention;

FIG. 20A depicts a an implant advanced via a delivery catheter into the coronary sinus according to an embodiment of the invention;

FIG. 20B shows the implant of FIG. 20A, wherein the distal anchor is deployed in the coronary sinus;

FIG. 20C depicts the implant of FIG. 20A, with the proximal implant being positioned at a desired location in the right atrium;

FIG. 20D depicts the implant of FIG. 20A, with the proximal anchor deployed in the right atrium;

FIG. 21 a side view of a three-anchor implant according to an embodiment of the invention;

FIG. 22 depicts a top view, in close-up, of a portion of a bridge according to an embodiment of the invention;

FIG. 23 depicts a side view of a three-anchor implant according to an embodiment of the invention;

FIG. 24 shows a three-anchor implant deployed in a heart according to an embodiment of the invention;

FIG. 25A shows a three-anchor implant deployed in a heart according to an embodiment of the invention;

FIG. 25B shows a three-anchor implant deployed in a heart according to an embodiment of the invention; and

FIG. 25C shows a three-anchor implant deployed in a heart according to an embodiment of the invention.

### DETAILED DESCRIPTION

Various embodiments of the present invention depict medical implants and methods of use that are well-suited for treating mitral valve regurgitation. It should be appreciated that the principles and aspects of the embodiments disclosed and discussed herein are also applicable to other devices having different structures and functionalities. For example, certain structures and methods disclosed herein may also be applicable to the treatment of other heart valves or other body organs. Furthermore, certain embodiments may also be used in conjunction with other medical devices or other procedures not explicitly disclosed. However, the manner of adapting the embodiments described herein to various other devices and functionalities will become apparent to those of skill in the art in view of the description that follows.

As used herein, “distal” means the direction of a device as it is being inserted into a patient's body or a point of reference closer to the leading end of the device as it is inserted into a patient's body. Similarly, as used herein “proximal” means the direction of a device as it is being removed from a patient's body or a point of reference closer to a trailing end of the device as it is inserted into a patient's body.

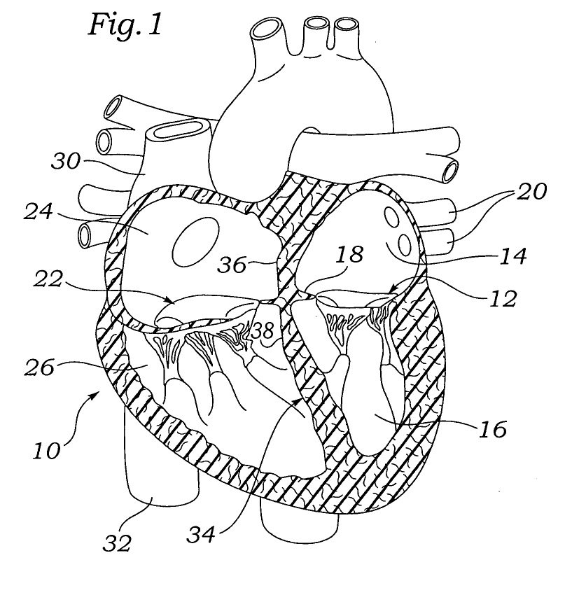

With reference now to FIG. 1, a four-chambered heart 10 is illustrated for background purposes. On the left side of the heart, the mitral valve 12 is located between the left atrium 14 and left ventricle 16. The mitral valve generally comprises two leaflets, an anterior leaflet and a posterior leaflet. The mitral valve leaflets are attached to a mitral valve annulus 18, which is defined as the portion of tissue surrounding the mitral valve orifice. The left atrium receives oxygenated blood from the pulmonary veins 20. The oxygenated blood that is collected in left atrium enters into the left ventricle through the mitral valve 12. Contraction of the left ventricle forces blood through the aortic valve and into the aorta.

On the right side of the heart, the tricuspid valve 22 is located between the right atrium 24 and right ventricle 26. The right atrium receives blood from the superior vena cava 30 and the inferior vena cava 32. The superior vena cava 30 returns de-oxygenated blood from the upper part of the body and the inferior vena cava 32 returns the de-oxygenated blood from the lower part of the body. The right atrium also receives blood from the heart muscle itself via the coronary sinus. The blood in the right atrium enters into the right ventricle through the tricuspid valve. Contraction of the right ventricle forces blood through the pulmonic valve and into the pulmonary trunk and then pulmonary arteries. The blood enters the lungs for oxygenation and is returned to the left atrium via the pulmonary veins 20.

The left and right sides of the heart are separated by a wall generally referred to as a septum 34. The portion of the septum that separates the two upper chambers (the right and left atria) of the heart is termed the atrial (or interatrial) septum 36 while the portion of the septum that lies between the two lower chambers (the right and left ventricles) of the heart is called the ventricular (or interventricular) septum 38.

On the left side of the heart, enlargement (i.e., dilation) of the mitral valve annulus 18 can lead to regurgitation (i.e., reversal of bloodflow) through the mitral valve 12. More particularly, when a posterior aspect of the mitral valve annulus 18 dilates, the posterior leaflet may be displaced from the anterior leaflet. As a result, the anterior and posterior leaflets fail to close completely and blood is capable of flowing backward through the resulting gap.

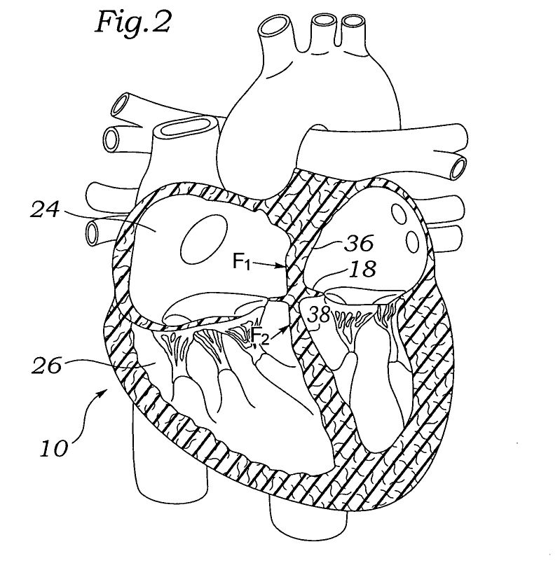

With reference now to FIG. 2, according to one aspect of the invention, a lateral force F₁ may be applied to the atrial septum 36 from within the right atrium 24 for altering the geometry of the mitral valve annulus on the left side of the heart. More particularly, the force applied along the atrial septum 36 may be used to reshape the mitral valve annulus 18. The resulting change in shape causes the anterior leaflet of the mitral valve to be located closer to the posterior leaflet. The effect of this is to close the gap between the leaflets. By closing the gap, leaflet coaption is improved, thereby reducing or eliminating mitral valve regurgitation. In addition or alternatively, a force F₂ may be applied to the ventricular septum 34 from within the right ventricle 26 to reshape the mitral valve annulus in a similar manner. In either case, it may be desirable that the force is applied to the septum at a location close to the mitral valve annulus.

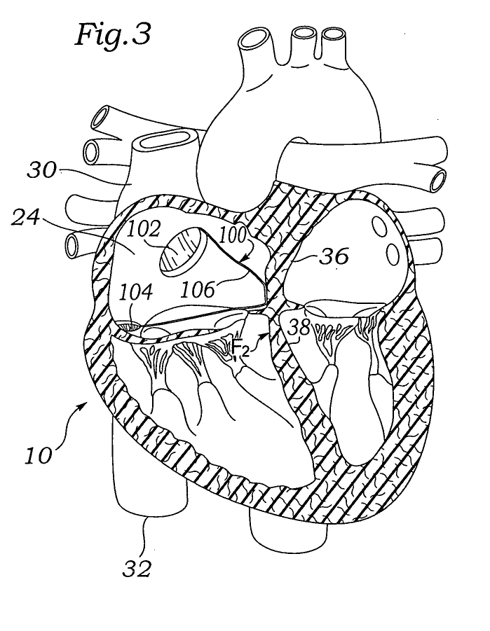

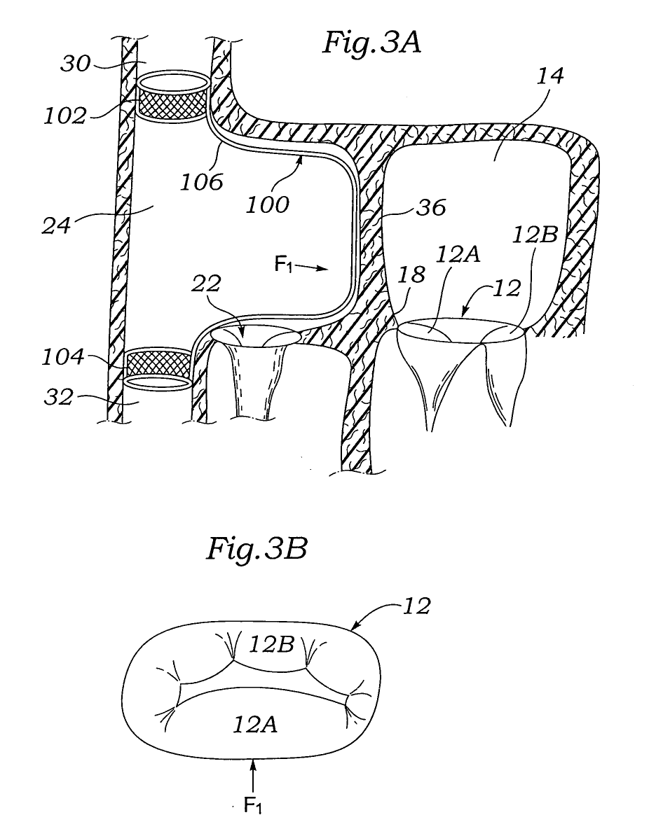

With reference now to FIGS. 3 through 3B, one embodiment of a mitral valve repair implant 100 is illustrated. The implant 100 is deployed substantially within the right atrium 24 and is configured to press against the atrial septum 36, which may occur along a lower portion of the atrial septum. One embodiment of the implant 100 comprises, generally, a first anchor 102, a second anchor 104 and a pusher member 106. The first anchor 102 may be an expandable stent configured to expand within the superior vena cava 30, which can be deployed along or adjacent to the ostium wherein the superior vena cava empties into the right atrium. The second anchor 104 may be an expandable stent configured to expand in the inferior vena cava 32, which can be deployed along or adjacent to the ostium wherein the inferior vena cava empties into the right atrium. The superior and inferior vena cava are desirable anchoring points because the tissue in this region is relatively stable and non-compliant and thereby provides a suitable foundation for anchoring the implant 100. Although the illustrated embodiment comprises two anchors, it will be appreciated that a device may be provided with only a single anchor while still remaining within the scope of the present invention.

The pusher member 106 can take the form of an elongate bridge extending between the first and second anchors. The pusher member may comprise a curved or bow-shaped wire configured for contacting the atrial septum 36. The implant may be formed of any suitable biocompatible material. In one embodiment, the pusher member 106 is formed at least in part from a shape memory material that bows outward after deployment. As illustrated, the pusher member may be shaped to extend along a path within the right atrium (e.g., along the wall) that minimizes adverse hemodynamic effects.

The pusher member 106 is configured for pushing against the atrial septum after the implant 100 has been deployed. In one embodiment, a resorbable material may be used to hold the pusher member in a contracted position during delivery and deployment. However, over time, the material is resorbed such that the pusher member is allowed to lengthen, thereby causing the pusher member to bow outward.

Resorbable materials are those that, when implanted into a human body, are resorbed by the body by means of enzymatic degradation and also by active absorption by blood cells and tissue cells of the human body. Examples of such resorbable materials are PDS (Polydioxanon), Pronova (Poly-hexafluoropropylen-VDF), Maxon (Polyglyconat), Dexon (polyglycolic acid) and Vicryl (Polyglactin). As explained in more detail below, a resorbable material may be used in combination with a shape memory material, such as Nitinol, Elgiloy or spring steel to allow the superelastic material to return to a predetermined shape over a period of time.

In the illustrated embodiment, the first and second anchors 102, 104 are both generally cylindrically shaped members. The first and second anchors 102, 104 each have a compressed state and an expanded state. In the compressed state, each of the first and second anchors has a diameter that is less than the diameter of the superior and inferior vena cava, respectively. In the expanded state, each of the first and second anchors has a diameter that is may be about equal to or greater than the diameter of the section of vena cava to which each anchor will be aligned. The anchors may be made from tubes of shape memory material, such as, for example, Nitinol. However, the anchors 102, 104 may also be made from any other suitable material, such as stainless steel. When the anchors are formed with stainless steel, the anchors may be deployed using a balloon catheter as known in the art. Although the anchor mechanisms take the form of stents for purposes of illustration, it will be appreciated that a wide variety of anchoring mechanisms may be used while remaining within the scope of the invention.

With particular reference to FIG. 3A, the functionality of the implant is schematically illustrated. It can be seen that the implant 100 is deployed in the right atrium 24 with the first anchor 102 expanded in the superior vena cava 30 and the second anchor 104 deployed in the inferior vena cava 32. The pusher member 106 extends between the anchors and is shaped for pressing against the atrial septum 36 for reshaping the mitral valve annulus 18 on the left side of the heart. In other words, the implant 100 applies a force F₁ against the atrial septum. With reference to FIGS. 3A and 3B, it can be seen that the force F₁ is transferred through the atrial septum for pushing the anterior leaflet 12A of the mitral valve 12 toward the posterior leaflet 12B.

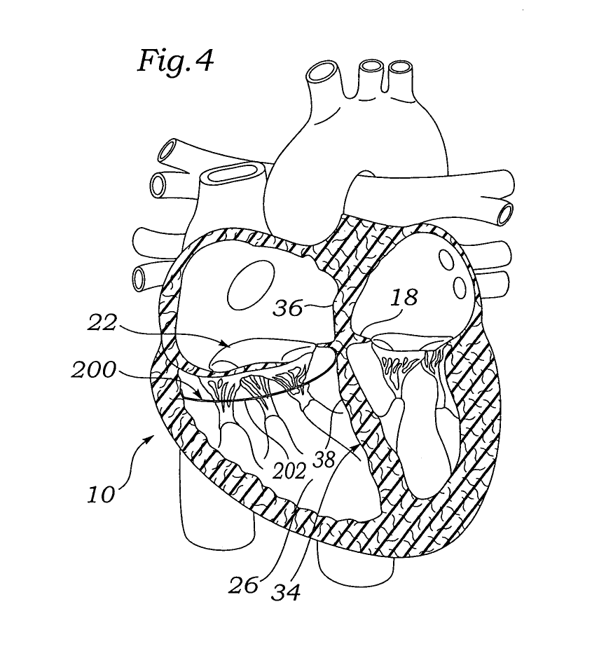

With reference now to FIG. 4, an alternative device 200 is illustrated for reshaping a mitral valve annulus. In this embodiment, the implant 200 is configured for deployment within the right ventricle 26. In one embodiment, the device generally comprises a U-shaped member 202 that is suitable for deployment in or adjacent to the tricuspid valve 22. More particularly, the U-shaped member may extend around the chordae and/or papillary muscles of the tricuspid valve. In a manner substantially similar to that described above, the U-shaped member urges the ventricular septum outward for reshaping the mitral valve annulus 18 and pushing the anterior leaflet of the mitral valve toward the posterior leaflet. Although a U-shaped member is shown for purposes of illustration, any suitable force applying member may be used.

Although particular devices have been illustrated for purposes of discussion, it will be appreciated that a variety of alternative mechanisms may be used to apply a force along the septum for reshaping the mitral valve annulus. For example, in one alternative embodiment, an expandable cage may be deployed in the right atrium for urging the atrial septum toward the left side of the heart, thereby moving the anterior leaflet toward the posterior leaflet. Still further, it will be appreciated that the devices and methods described herein may also be used to treat the tricuspid valve. Those skilled in the art will appreciate that a substantially similar device may be deployed in the left atrium (or left ventricle) for pushing the septum toward the right side of the heart and improving coaption of the tricuspid leaflets.

To further enhance the ability to reshape the mitral valve annulus, an implant for pushing against the anterior leaflet of the mitral valve, such as the embodiments described above, may be used in combination with an implant deployed in the coronary sinus for pushing against the posterior leaflet of the mitral valve. One example of a device configured for deployment in the coronary sinus is described in Applicant's co-pending application Ser. No. 11/238,853, filed Sep. 28, 2005, the contents of which are hereby incorporated by reference. It will be recognized that, by applying compressive forces to both the anterior and posterior sides of the mitral valve, the ability to improve leaflet coaption is further enhanced.

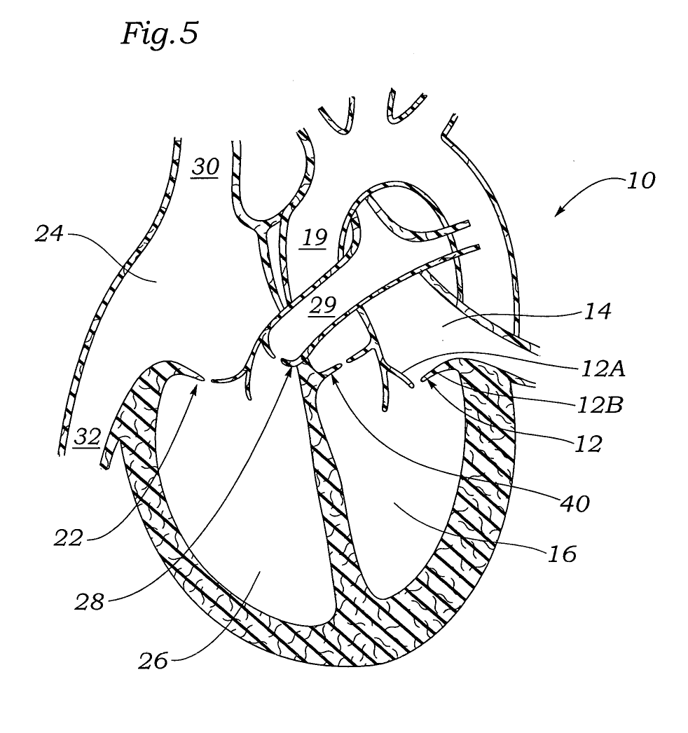

With reference now to FIG. 5, an alternative illustration of a four-chambered heart 10 is provided wherein all four heart valves can be seen. As discussed above, on the left side of the heart, the mitral valve 12 is located between the left atrium 14 and left ventricle 16. The mitral valve generally comprises two leaflets, an anterior leaflet 12A and a posterior leaflet 12B. Contraction of the left ventricle forces blood through the left ventricular outflow tract (LVOT) and into the aorta 19. The aortic valve 40 is located between the left ventricle 16 and the aorta 19 for ensuring that blood flows in only one direction (i.e., from the left ventricle to the aorta). As used herein, the term left ventricular outflow tract, or LVOT, is intended to generally include the portion of the heart through which blood is channeled from the left ventricle to the aorta. The LVOT shall include the aortic valve annulus and the adjacent region extending below the aortic valve annulus. For purposes of this discussion, the LVOT shall also include the portion of the ascending aorta adjacent to the aortic valve.

On the right side of the heart, the tricuspid valve 22 is located between the right atrium 24 and right ventricle 26. The right atrium receives blood from the superior vena cava 30 and the inferior vena cava 32. Contraction of the right ventricle forces blood through the right ventricular outflow tract (RVOT) and into the pulmonary arteries. The pulmonic valve 28 is located between the right ventricle and the pulmonary trunk 29 for ensuring that blood flows in only one direction from the right ventricle to the pulmonary trunk. As used herein, the term right ventricular outflow tract, or RVOT, generally includes the pulmonary valve annulus and the adjacent region extending below the pulmonary valve annulus.

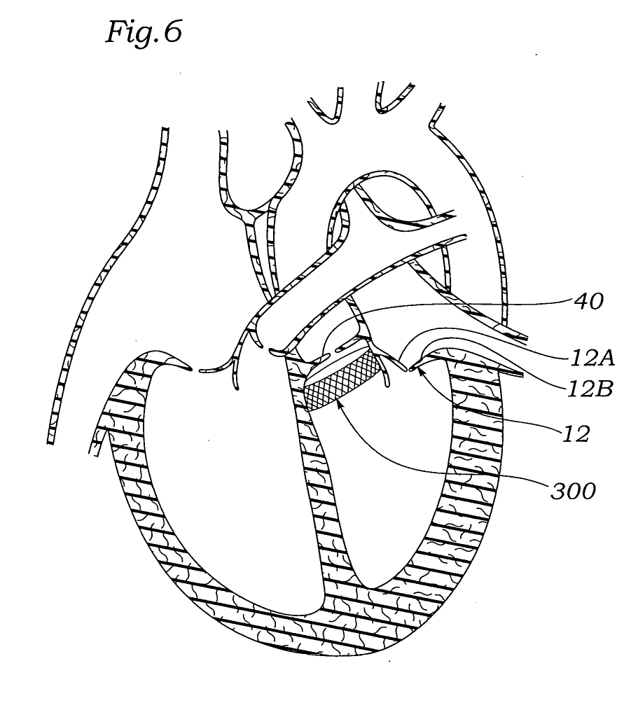

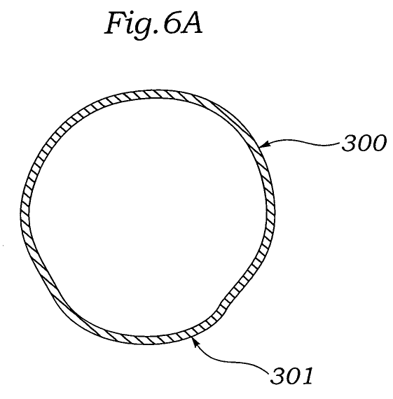

With reference now to FIG. 6, another embodiment of a medical implant 300 is illustrated for treating mitral valve regurgitation. In this embodiment, the implant 300 is configured for deployment within the LVOT at a location beneath the aortic valve. Due to the proximity of the LVOT with respect to the anterior portion of the mitral valve annulus, it has been found that the deployment of an implant within the LVOT may be used to reshape the mitral valve annulus and thereby affect the position of the anterior leaflet of the mitral valve. More particularly, the implant is configured to apply a force which pushes the anterior leaflet 12A toward the posterior leaflet 12B for improving leaflet coaption in the mitral valve.

In one embodiment, the implantable device 300 generally comprises an expandable stent. The stent may be self-expanding or balloon-expandable. When a self-expanding stent is used, the stent may be formed of a shape memory material and may be delivered using a sheath. After reaching the treatment site, the stent is emitted from the sheath and is allowed to self expand. When a balloon-expandable stent is used, the stent may be formed of stainless steel. The stent is crimped and placed over a deflated balloon provided on the distal end portion of an elongate catheter. The distal end portion of the catheter is advanced to the treatment site and the balloon is inflated for expanding the stent within the LVOT. If desired, the stent may further comprise engagement members, such as, for example, barbs or hooks, to enhance the securement of the stent at the treatment site. As shown in FIG. 6A, if desired, the stent may be formed with a bulge or protrusion 301 for increasing the force applied in the region of the anterior leaflet.

The implant 300 may be delivered to the treatment site using a minimally invasive procedure. In one method of use, the device is inserted through the femoral artery and is advanced around the aortic arch to the treatment site. In another method of use, the device is inserted into the femoral vein and is advanced from the right side of the heart to the left side of the heart via a trans-septal procedure. After reaching the left side of the heart, the device can be deployed within the LVOT.

The implant 300 may be configured to expand to a diameter greater than the natural diameter of the LVOT. As a result of the expansion, an outward force is applied along the LVOT. More particularly, a force is applied along a region of tissue adjacent the anterior portion of the mitral valve. The force urges the anterior leaflet toward the posterior leaflet of the mitral valve for reducing or eliminating mitral valve regurgitation.

The device may be used alone or in combination with another therapeutic device, such as an implant configured for deployment within the coronary sinus. When used with an implant in the coronary sinus, compressive forces may be applied along both the anterior and posterior portions of the mitral valve, thereby providing the clinician with an enhanced ability to improve leaflet coaption and reduce mitral valve regurgitation.

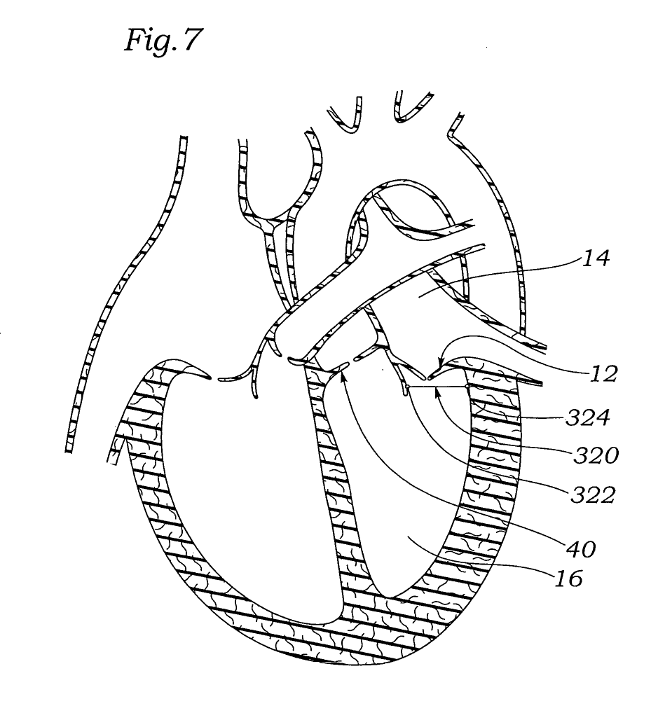

With reference to FIG. 7, yet another device and method for treating mitral valve regurgitation is schematically illustrated. In this embodiment, a tether 320 or other tension member extends across a portion of the left ventricle for pulling the anterior and posterior mitral valve leaflets together. The tether may take the form of a suture which is passed through tissue along the walls of the left ventricle. One device for deploying a suture or tether can be found in Applicant's co-pending application Ser. No. 10/389,721, filed Mar. 14, 2003, now published as U.S. Publication No. 2004/0181238, the contents of which are hereby incorporated by reference. In an alternative device, the tether may have barbs or other anchoring means for engaging the tissue. If necessary, more than one tether may be used for reshaping the mitral valve annulus and improving leaflet coaption.

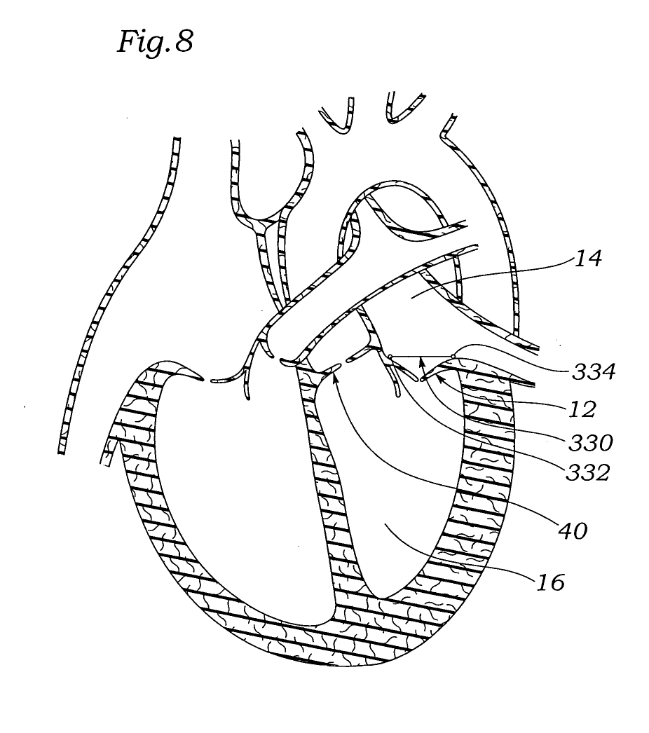

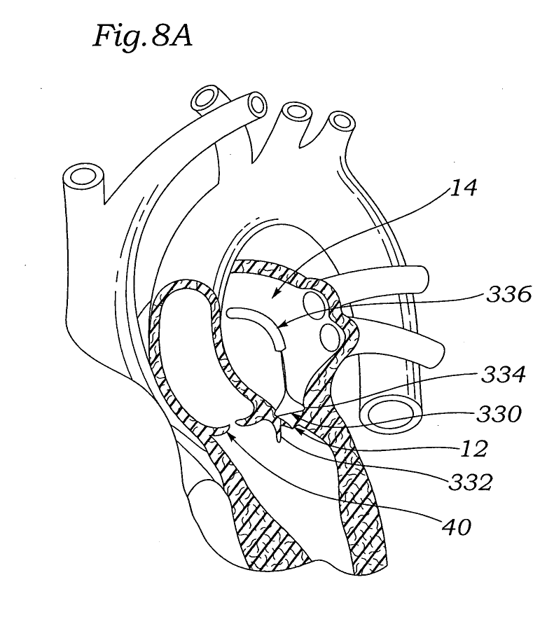

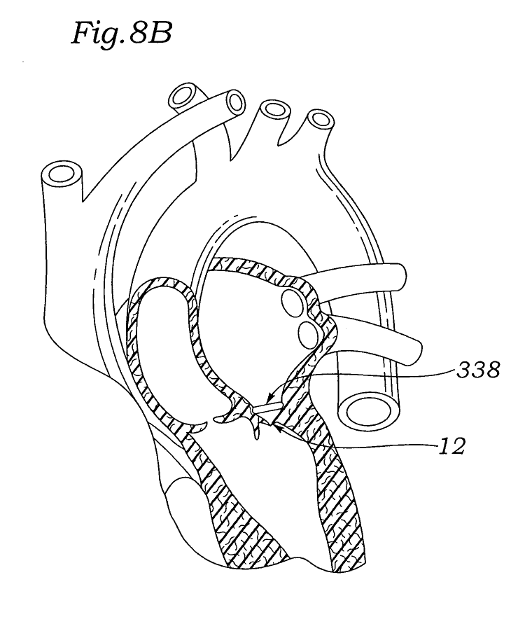

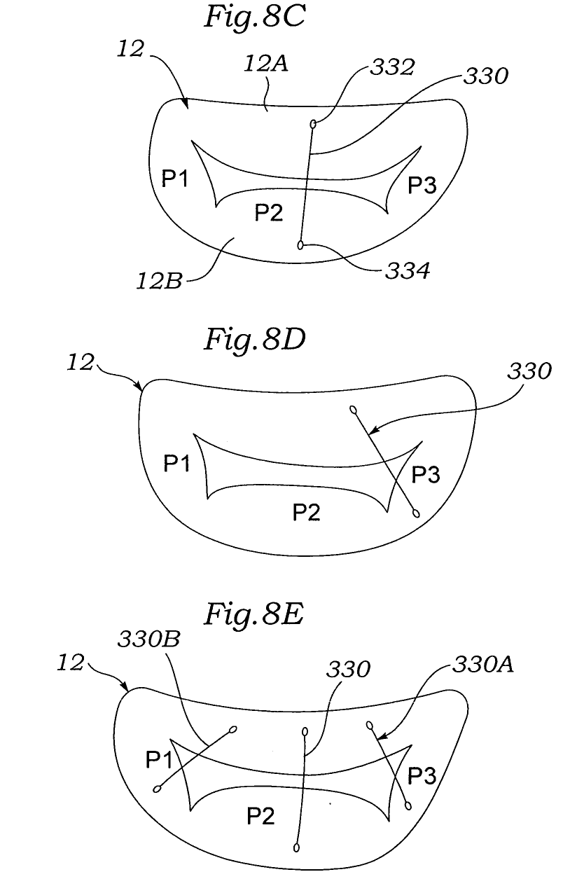

With reference to FIG. 8, yet another alternative approach is schematically illustrated for treating the mitral valve. In this embodiment, a tether 330 or other elongate tension member extends across a portion of the left atrium for pulling the anterior and posterior mitral valve leaflets together. The tether may be attached to opposing regions of tissue on the mitral valve annulus. The tether may take the form of a suture which is tied or otherwise fastened to the tissue along the mitral valve annulus.

In one method of delivering the tether, a repair device is provided which has a deployment mechanism for applying first and second fastener elements to first and second regions of the mitral valve annulus. The first region of tissue is grasped using the repair device and the first fastener element 332 is deployed into the first region of tissue. The first region of tissue is disengaged from the repair device while leaving the first fastener element deployed therein. The second region of tissue is then grasped using the repair device and the second fastener element 334 is deployed into the second region of tissue. The second region of tissue is disengaged from the repair device while leaving the second fastener element deployed therein. The first and second fastener elements are attached by the tether 330. The tether pulls the first and second fastener elements together for reducing the distance between the first and second regions of tissue, thereby reshaping the mitral valve annulus. The tether is held in tension for maintaining the mitral valve annulus in the reshaped condition.

With reference to FIG. 8A, a more particular method of use will be described in more detail. In this method, a distal end portion of a therapy catheter 336 is percutaneously advanced into the left atrium 14. The therapy catheter may include a side vacuum port (not shown) for grasping tissue. After grasping the tissue on one side of the mitral valve annulus, a needle is advanced from the catheter and through the tissue for advancing a first piece of suture through the tissue. The tissue is then released and the procedure is repeated on the other side of the annulus, thus creating a suture loop. As best shown in FIG. 8B, a clip or other fastener 338 is then advanced over the suture to hold the loop tight and the remaining suture is cut away and removed. The suture loop and clip provide the tether for maintaining the mitral valve annulus in the reshaped condition.

With reference to FIG. 8C, a mitral valve 12 is illustrated wherein a tether 330 has been secured to opposite sides of the mitral valve annulus along a central region of the mitral valve. The tether is attached with sufficient tension such that the mitral valve annulus is reshaped for improving coaption between the anterior leaflet 12A and posterior leaflet 12B. FIG. 8D illustrates an alternative approach wherein a tether 330A is secured to the posterior portion of the mitral valve annulus adjacent to a P3 scallop. FIG. 8E illustrates another alternative configuration wherein a plurality of tethers 330, 330A, 330B are provided. These various approaches are provided for purposes of illustration; however, it will be appreciated that a variety of alternative approaches may also be selected for treating a particular defect.

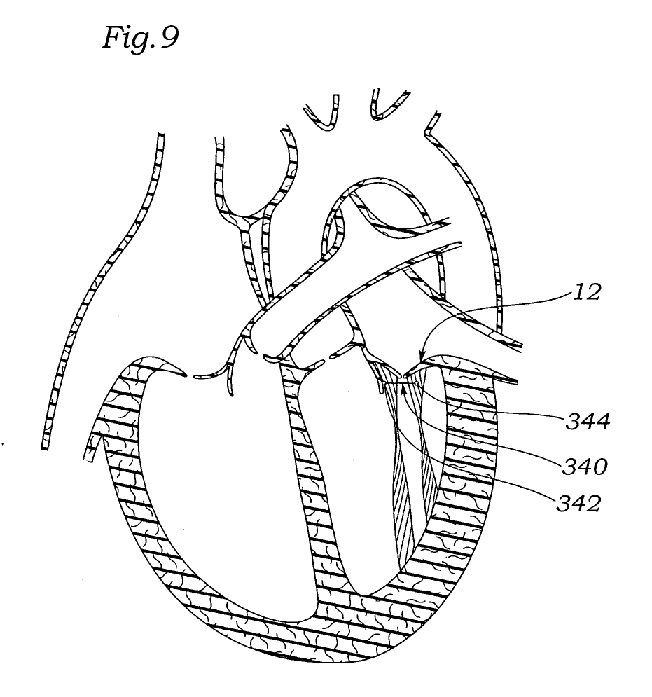

With reference to FIG. 9, another embodiment of a tether 340 is illustrated wherein at least one end of the tether is configured for attachment to chordae.

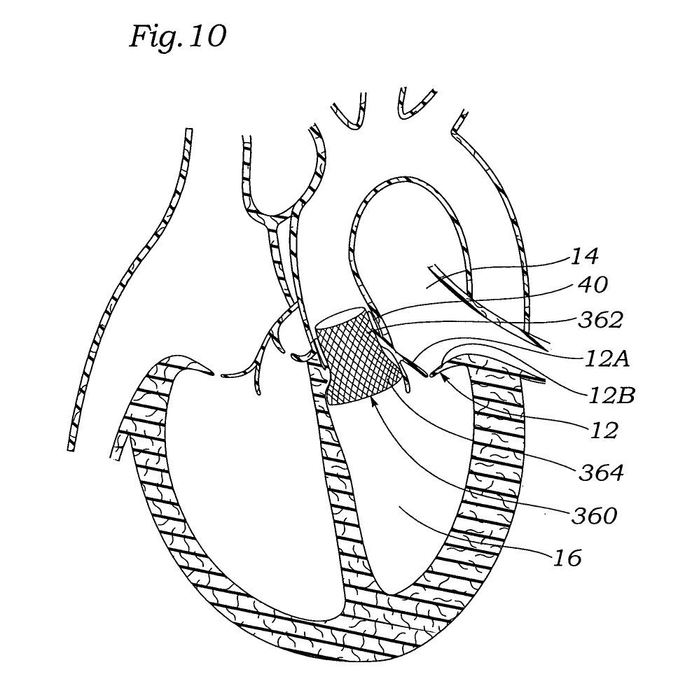

With reference to FIG. 10, yet another approach for treating mitral valve regurgitation comprises a prosthetic valve 360 configured for deployment within the aortic valve annulus. The prosthetic valve may includes an expandable stent portion and a valvular structure disposed within the stent portion. The prosthetic valve is configured to replace the function of the native aortic valve 40. The stent portion of the prosthetic valve is configured to extend below the aortic valve annulus and into the LVOT. The stent is shaped to apply a force along the region of tissue which separates the LVOT from the mitral valve. The force moves the anterior leaflet 12A of the mitral valve 12 toward the posterior leaflet 12B for improving leaflet coaption. In one configuration, the stent portion includes a generally tubular upper section which contains the valvular structure. If desired, the stent portion may include a flared lower portion 364 configured to engage and push against the tissue of the LVOT, thereby more effectively altering the position of the anterior leaflet 12A. This embodiment advantageously provides the clinician with the ability to treat both the aortic valve and the mitral valve with a single device. Addition details regarding the structure and use of prosthetic valves can be found in Applicant's U.S. Pat. No. 6,730,118, the contents of which are hereby incorporated by reference.

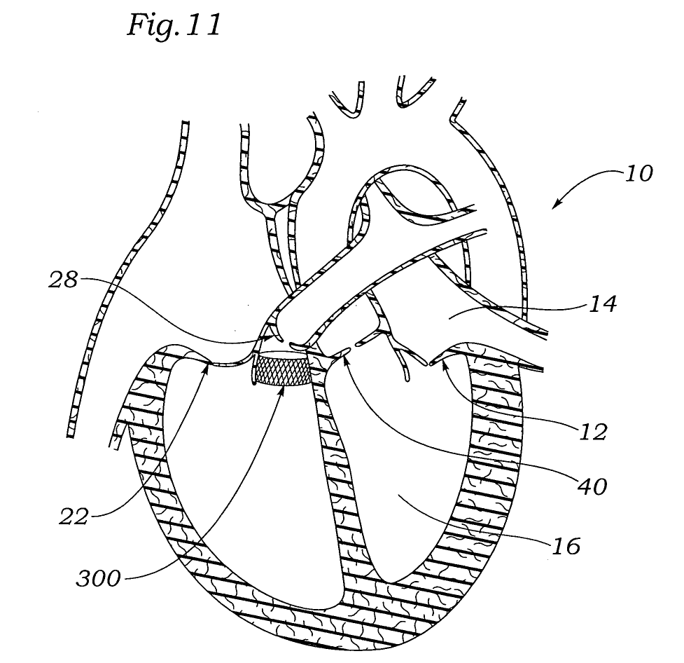

It will be recognized that the embodiments described above may also be used to treat a triscuspid valve in substantially similar manner. For example, with reference to FIG. 11, in an approach similar to that described with respect to FIG. 6, an expandable stent 300 may be deployed in the RVOT for pushing against the anterior region of the tricuspid valve. Depending on the particular anatomy, this method may be used to advantageously treat tricuspid valve regurgitation. Furthermore, aspects of each of the other embodiments described herein may also be used to treat the triscuspid valve.

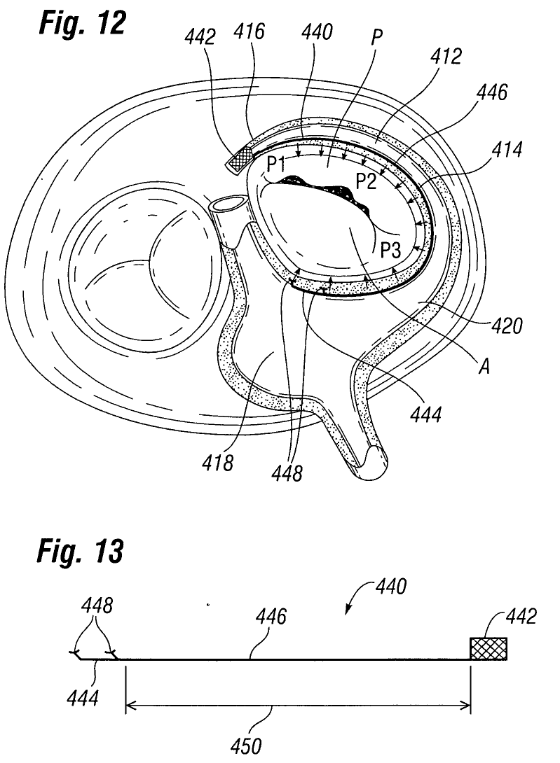

FIG. 12 depicts an implant 440 according to an embodiment of the current invention deployed in the coronary sinus 412 and right atrium 418 of a mitral valve 414. As depicted in FIG. 12, the implant 440 of the invention applies tension not only within the coronary sinus 412 but also to portions of the right atrium 418, thereby engaging against the atrial septum while pulling one or more portions of the coronary sinus 412 into a more straightened (i.e., less curved or dilated) configuration, which creates a corresponding reshaping of the posterior aspect of the mitral valve annulus 422. The implant 440 thus causes movement of the posterior aspect of the mitral valve annulus 422 in an anterior direction, thereby moving the posterior leaflet P (and its scallops P1, P2, P3) closer to the anterior leaflet A and closing the gap caused by the leaflet displacement.

The implant 440 includes a distal anchor 442, a proximal anchor 444, and a connecting bridge 446. The distal anchor 442 is depicted deployed in a generally narrow portion of the coronary sinus 412, while the proximal anchor 444 is deployed in the right atrium 418. In the particular embodiment depicted in FIG. 12, the proximal anchor 444 includes a plurality of barbs 448 configured to be attached to tissue of the right atrium 418. In the particular embodiment depicted in FIG. 12, the proximal anchor 444 is secured to atrial tissue lying generally between the ostium 420 and superior vena cava 458. The connecting bridge 446 pulls the distal and proximal anchors 442, 444 toward each other, thereby changing the curvature of the coronary sinus 412 and moving the posterior leaflet P (with scallops P1, P2, P3) toward the anterior leaflet A.

The implant 440 of the invention, and/or one or more parts thereof (e.g., the distal anchor 442, proximal anchor 444, and/or bridge 446) can be formed from various biocompatible materials, such as metals, plastics, bioresorbable materials, etc. A shape memory material such as Nitinol may be used for one or more elements, with appropriate biasing toward or away from the use/deployed configuration, so as to provide self-expandable, self-deploying, self-shortening, or other function to one or more parts of the implant 440.

FIG. 13 depicts the device 440 by itself in a straightened configuration to better present aspects of the device 440. The bridge 446 connects the distal and proximal anchors 442, 444. The bridge 446 defines a length 450 between the distal and proximal anchors 442, 444. Depending on the particular embodiment, the bridge 446 may be adapted to selectively vary the length 450. For example, the bridge 446 may be configured to reduce its length 450 via the use of memory metals, resorbable materials, etc. The bridge 446 may be adapted to be threaded with a resorbable material, such as a coil or X-shape bridge structure threaded with resorbable thread. Resorbable materials are those that, when implanted into a human or other animal body, are resorbed by the body by means of enzymatic degradation and/or by the active absorption by blood and tissue cells of the body. The bridge 446 may also or alternatively be slidingly or otherwise adjustingly disposed with respect to one or more of the anchors 442, 444, so that one or more of the anchors 442, 444 can be slidingly advanced (or otherwise moved) along the material forming the bridge 446 toward or away from the opposing anchor. Bridge lengths for use with the invention may range from 40 mm to 150 mm, depending on the particular application. These and other bridges having various configurations as are generally known in the art are within the scope of the invention.

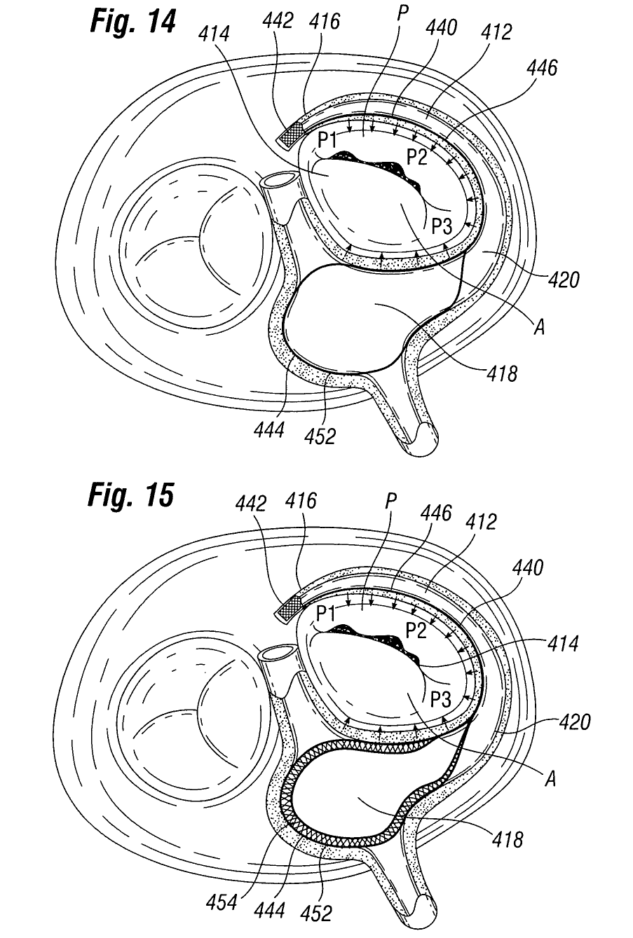

FIG. 14 depicts a further embodiment of the invention, wherein an implant 440 includes a distal anchor 442 deployed in a distal portion 416 of the coronary sinus 412, a proximal anchor 444 deployed in the right atrium 418, and a connecting bridge 446. The proximal anchor 444 comprises a loop 452 of material passing around a perimeter of the right atrium 418. The loop 452 of the proximal anchor may be self-expanding, or may be balloon-expandable, or otherwise deployable from a delivery configuration to the deployed loop configuration depicted in FIG. 14.

FIG. 15 depicts a further embodiment of the invention, wherein an implant 440 includes a distal anchor 442 deployed in a distal portion 416 of the coronary sinus 412, a proximal anchor 444 deployed in the right atrium 418, and a connecting bridge 446. The proximal anchor 444 comprises a loop 452 of expandable stent mesh anchor structure 454 passing around a perimeter of the right atrium 418. The stent mesh anchor structure 454 may be self-expanding and biased toward the deployed configuration.

The bridge 446 and/or anchors 442, 444, including the loop 452 and stent mesh anchor structure 454, may be formed from a shape memory metal such as Nitinol, or from other materials such as stainless steel, other metals, plastic, etc. The materials of the anchors 442, 444 and bridge portion 446 may preferably be biocompatible.

The anchors 442, 444 and/or bridge 446 may include one or more visualization references. For example, visualization references in the form of radiopaque marker bands may be positioned on or adjacent the distal and proximal anchors respectively. The radiopaque marker bands are viewable under a fluoroscope, so that a surgeon or other user can use a fluoroscope to visualize the position of the anchors within the patient and with respect to any delivery catheter or other delivery devices present, such as guidewires, etc. Depending on the particular application, the visualization markers on a particular implant may be identical or may be different from each other. Radiopaque marker bands or other visualization references that provide different radiopaque or other visualization signatures permit a user to differentiate between particular elements of a particular implant. For example, different radiopaque signatures from a distal anchor marker band and a proximal anchor marker band would permit the user to distinguish between the distal anchor and proximal anchor, and thus better visualize the location and orientation of the implant when viewing the implant in a patient's body under fluoroscopy.

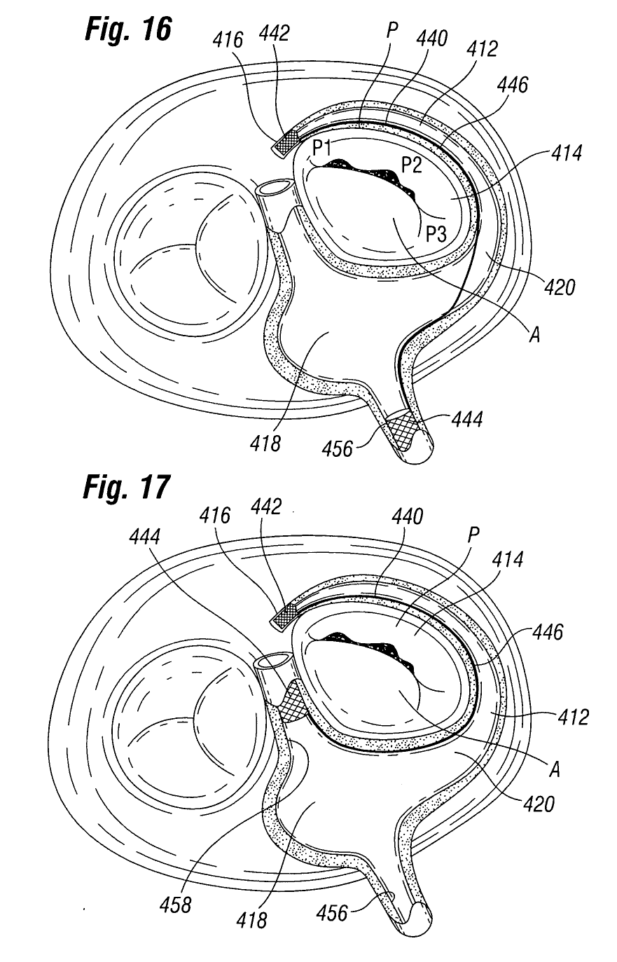

FIG. 16 depicts a further embodiment of the invention, wherein the device 440 has a distal anchor 442 deployed in a distal portion 416 of the coronary sinus 412, and a proximal anchor 444 deployed in the inferior vena cava 456, with a bridge element 446 connecting the anchors 442, 444.

FIG. 17 depicts a further embodiment of the invention, wherein a device 440 has a distal anchor 442 deployed in a distal portion 416 of the coronary sinus 412, a proximal anchor 444 deployed in the superior vena cava 458, and a bridge element 446 connecting the anchors 442, 444 and engaging against the atrial septum. Such deployment provides for an almost complete encirclement of the annulus 422 of the mitral valve 414.

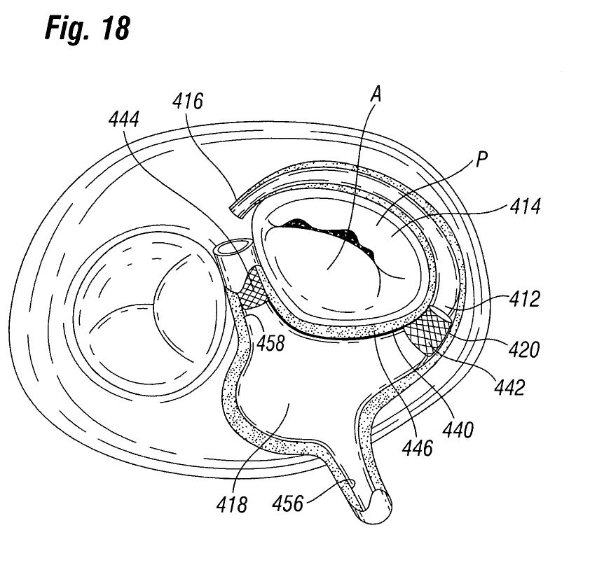

While the above embodiments depict the distal anchor deployed in a distal area of the coronary sinus, the distal anchor could be deployed elsewhere, depending on the particular application, including the (pre-treatment) shape of the mitral valve and desired reshaping to be achieved. For example, a distal anchor could be deployed in the coronary sinus adjacent the P1, P2, or P3 leaflets. In one particular embodiment depicted in FIG. 18, the distal anchor 442 is deployed in a proximal portion of the coronary sinus 412 (e.g., just inside of the ostium 420), with the proximal anchor 444 positioned within the superior vena cava 458.

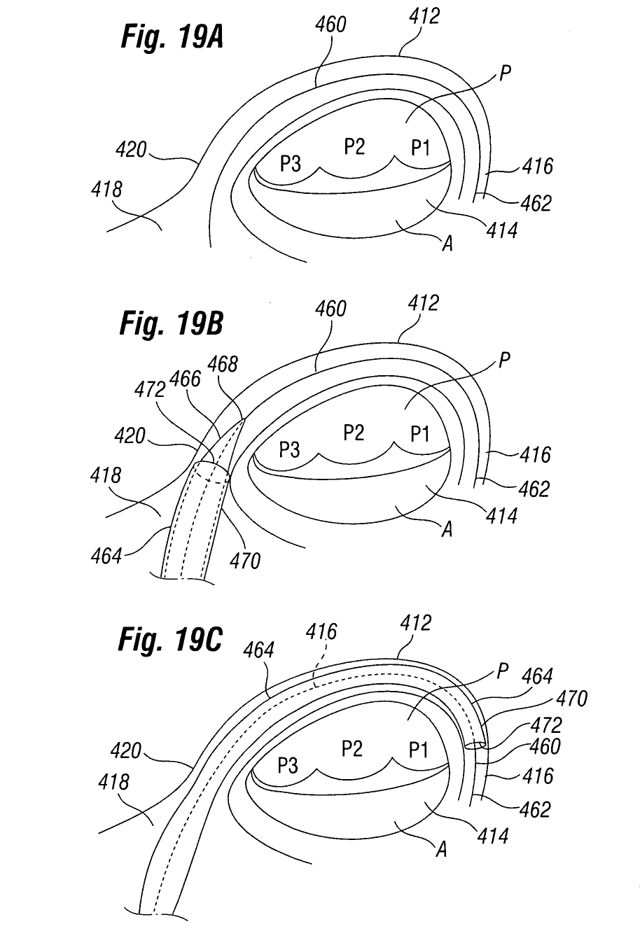

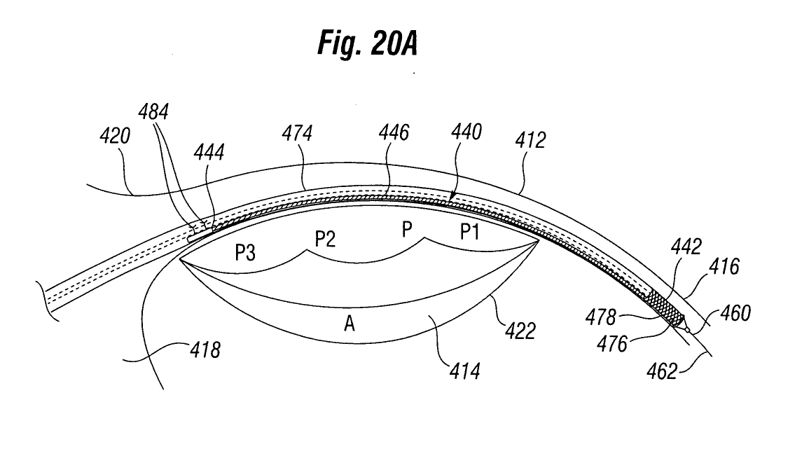

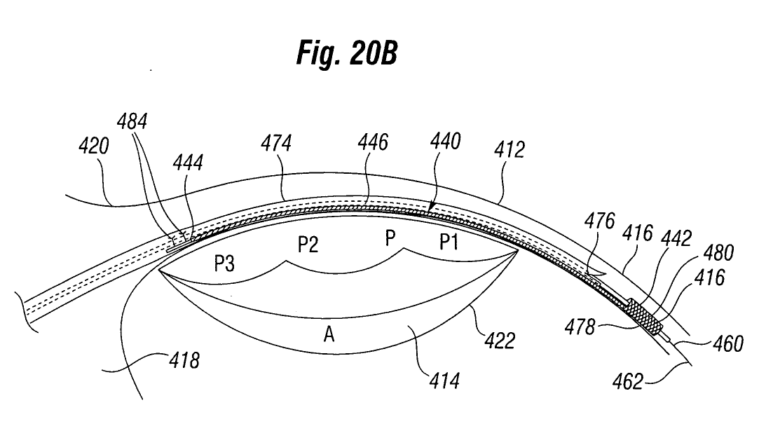

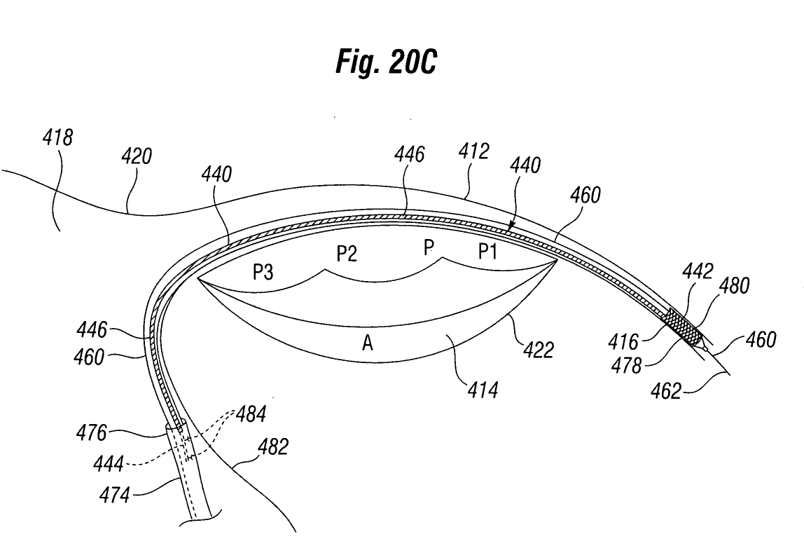

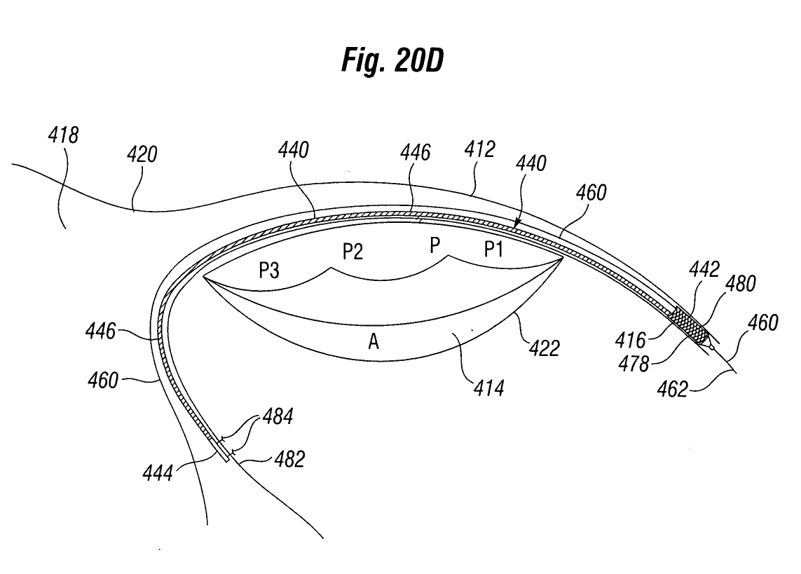

The device can be deployed according to various methods. One particular method is depicted in FIGS. 19A-19C and 20A-20D. First, the patient is prepared and an introducer sheath (not shown) may be inserted into a left or right internal jugular vein or the femoral vein to provides access to the coronary sinus as is generally known in the art. Once the introducer sheath is secured, a guidewire 460 is inserted through the introducer sheath and into the coronary sinus 412 (i.e., into the great cardiac vein), as shown in FIG. 19A. With the distal end 462 of the guidewire 460 advanced distally into the coronary sinus 412, a guide catheter 464 and dilator 466 may then be inserted. In preparation for deployment of the catheter 464 and dilator 466, a syringe filled with flushing fluid, such as heparinized saline solution, may be used to flush the dilator 464 and/or the guide catheter 466 to remove any residual air and improve lubricity. A hemostatic valve (not shown), such as a Y-connector, may attached to a proximal end of the guide catheter 464. The hemostatic valve minimizes blood loss through an interface between the guide catheter 464 and other devices (such as the dilator 466) loaded through the guide catheter 464.

As depicted in FIG. 19B, the dilator 466 is positioned in a central lumen of the guide catheter 464 such that the tapered distal portion 468 of the dilator 466 extends out of the guide catheter distal end 470 via a distal opening 472. The dilator distal portion 468 serves to provide a smooth transition between the relatively small diameter guidewire 460 and the relatively large diameter guide catheter 464, thereby reducing the chance that a leading edge of the guide catheter 466 (e.g., an edge of the guide catheter distal end 470) will engage against body lumen walls or other tissue as the guide catheter 464 is inserted. As shown in FIG. 19B, the guide catheter 464 and dilator 466 are placed onto the guidewire 460 and advanced over the guidewire 460 until the distal end 470 of the guide catheter 464 is position at a desired location in the coronary sinus 412. In the particular embodiment depicted in FIGS. 19B and 19C, the guide catheter distal end 470 is positioned adjacent the ostium 420 of the coronary sinus 412. The process may be monitored via fluoroscopy and/or other viewing methods.

With the guide catheter distal end 470 positioned at the desired location (e.g., in a distal portion 416 of the coronary sinus 412, or just in or adjacent the ostium 420), the dilator 466 can be withdrawn proximally from the guide catheter 464, with the guide catheter distal end 470 remaining in the desired location as the dilator 466 is withdrawn. The guide catheter 464 will remain in the desired position, as depicted in FIG. 19C, to serve as a guide within which additional devices (e.g., treatment/deployment/delivery catheters, etc.) may subsequently be advanced to within the coronary sinus and/or other deployment locations. Note that use of the guidewire 460, guide catheter 462, and dilator 464 are optional, and their use depends on the particular application.

With a guide catheter secured (if present) at a desired location, a delivery catheter 474 can be inserted over the guidewire 460 and advanced into in the coronary sinus 412, as depicted in FIG. 20A. The delivery catheter 474 contains the implant 440, and is advanced until the distal anchor 442 of the implant 440 (which in the particular embodiment depicted is positioned within a distal end 476 of the delivery catheter 474) is adjacent a distal anchor desired deployment location 478, which in the embodiment depicted is in a distal portion 416 of the coronary sinus 412. Note that the guide catheter 464 from FIGS. 19A-19C is not depicted in FIGS. 20A-20E, although it may be used depending on the particular application.

Once the distal anchor 442 is positioned adjacent the distal anchor desired deployment location 478, the distal anchor 442 is deployed to be secured at the distal anchor desired deployment location 478, as depicted in FIG. 20B. In the particular embodiment depicted, the distal anchor 442 is an expandable mesh stent 480, which can be configured to be self-expanding (e.g., formed from Nitinol, etc.) or balloon-expandable (e.g., formed from stainless steel, etc.) to form a generally tubular structure that engages against the walls of the coronary sinus 412.

Referring now to FIG. 20C, after the distal anchor 442 is deployed, the proximal anchor 444 is positioned at a proximal anchor desired deployment location 482, which in the particular embodiment depicted is within the right atrium 418. The particular proximal anchor 444 depicted is a series of barbs 484 configured to engage against tissue of the right atrium 418. As the proximal anchor 444 is brought to the proximal anchor desired deployment location 482, the movement of the (as-yet-undeployed) proximal anchor 444 with respect to the already-deployed distal anchor 442 creates tension in the bridge 446, thereby pulling the distal anchor 442 (and coronary sinus distal portion 416) proximally and causing a reshaping of the mitral valve annulus 422.

With the proximal anchor 444 positioned at or adjacent the proximal anchor desired deployment location 482, the proximal anchor 444 is deployed, which in the particular embodiment depicted in FIG. 20D involves deploying the proximal anchor barbs 484 to engage against tissue of the right atrium 418. The delivery catheter 474 is then removed from the patient.

Note that the particular order of deployment depends on the particular application, including issues such as the desired deployment sites for the distal and proximal anchors, the configuration of the implant, and the nature of the bridge, e.g., fixed length, immediately-adjustable length (e.g., via ratchets, etc.), and/or slowly-adjustable length (memory metal, dissolving portions, etc.). For example, in other embodiments, the proximal anchor could be deployed prior to deployment of the distal anchor, or the distal anchor and proximal anchor could be deployed generally simultaneously.

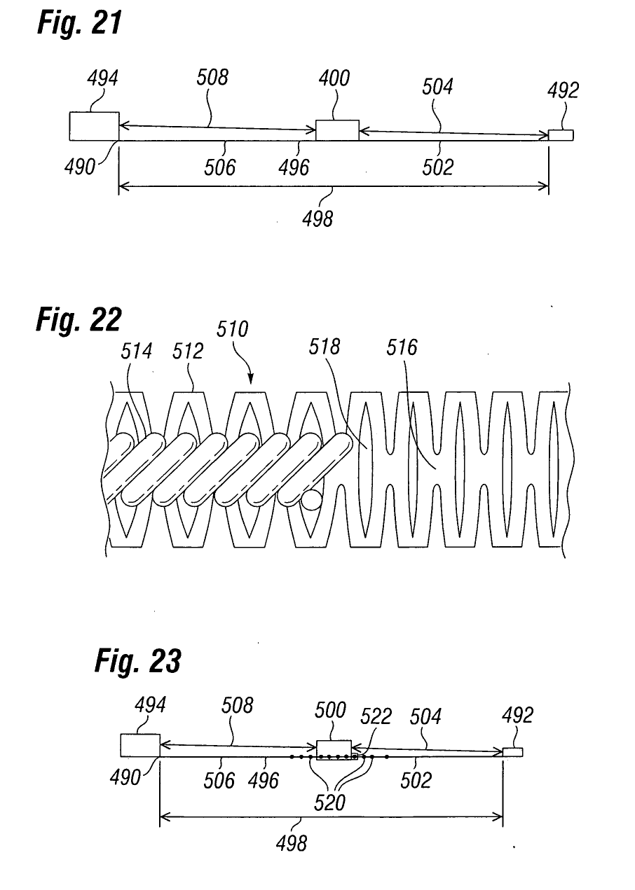

FIG. 21 is a top view of an implant 490 according to a further embodiment of the invention. The implant 490 has a distal anchor 492, proximal anchor 494, and bridge 496 defining a bridge length 498 between the distal anchor 492 and proximal anchor 494. The implant 490 also includes a middle anchor 500 positioned along the bridge 496 between the distal anchor 492 and proximal anchor 494. A distal bridge section 502 defines a distal bridge length 504 between the middle anchor 500 and the distal anchor 492, and a proximal bridge section 506 defines a proximal bridge length 508 between the middle anchor 500 and the proximal anchor 494.

As was the case with the two-anchor implant discussed previously with respect to FIGS. 12-20D, the bridge length 498, distal bridge length 504, and/or proximal bridge length 508 of the three-anchor implant 490 may be fixed between the respective anchors, or may be adjustable. Adjustable lengths can be achieved through delayed shortening or lengthening via the use of memory materials and/or bio-resorbable materials, and/or the respective anchors may be configured to be advanced (via ratcheting or similar configuration) in one direction or another along the bridge 496 and/or bridge sections 502, 506. For example, the middle anchor 500 may be configured to be advanced in one or another direction along the bridge 496, such as by a ratcheting adjustment. The distal bridge length 504 and/or proximal bridge length 508 may be also adjustable via various methods.

The proximal, middle, and distal anchors may be used with bridges and bridge sections having various structures as are generally known in the art. The bridge and bridge sections serve to separate the various anchors by a desired distance and may also serve to reduce the distance between the anchors when the implant is inserted into the coronary sinus, thus allowing the implant to reduce mitral regurgitation. The bridge may be adapted to be acutely cinchable, or it may be adapted for delayed release.

An example of a bridge and/or bridge portions configured for delayed shortening involves a coil-like or lattice-like bridge structure threaded with a resorbable material such as resorbable suture. Resorbable materials are those that, when implanted into a human body, are resorbed by the body by means of enzymatic degradation and/or by active absorption by blood cells and tissue cells of the human body. Examples of such resorbable materials include resorbable metals, such as magnesium alloys and zinc alloys, and resorbable polymers such as PDS (Polydioxanon), Pronova (Poly-hexafluoropropylen-VDF), Maxon (Polyglyconat), Dexon (polyglycolic acid), and Vicryl (Polyglactin). A resorbable material may be used in combination with a shape memory material, such as Nitinol, Elgiloy, or spring steel to allow the superelastic material to return to a predetermined shape over a period of time.

In the example of FIG. 22, a bridge portion 510 (which can form all or a portion of the proximal and/or distal bridge portions 504, 508 of an implant 490 such as that depicted in FIG. 21) has a non-resorbable spring-like structure 512 threaded with resorbable material 514, and more specifically includes “X”-shaped bridge elements 516 with resorbable material 514 passing through openings 518 therein. The spring-like structure 512 of the bridge portion 510 will contract over time as the resorbable material 514 is absorbed into the body. Such an embodiment is described in U.S. patent application Ser. No. 11/014,273, entitled “Device for Changing the Shape of the Mitral Annulus” and filed on Dec. 15, 2004, the entire contents of which are incorporated herein.

Referring now to FIG. 23, an implant 490 includes a distal anchor 492, proximal anchor 494, middle anchor 500, and distal and proximal bridge portions 504, 508 forming a generally continuous bridge 496. The bridge 496 is adapted to provide acute cinching. The bridge 496 includes obstructions in the form of knots 520 which may be pulled through a lock 522 positioned on or in the middle anchor 500. The lock 522 may adapted to allow the knots 520 (or similar structures, such as extensions or indentations) on the bridge 496 to pass through in one direction but to prevent the knots 520 from passing back through in an opposite direction. For example, the lock 522 may be configured in one embodiment to prevent the knots from passing distally through the lock 522, or in another embodiment from passing proximally through the lock 522. The lock 522 may also be configured to have an open configuration and a closed configuration. For example, in an open configuration the lock may permit the knots to pass therethrough in either direction (distally or proximally), but in the locked position may be configured to prevent distal and/or proximal passage of the knots 520. The number of knots 520 and the spacing between the knots 520 may vary according to preferences. The distance between the middle anchor 500 and distal anchor 492 and/or proximal anchor 494 can thus be adjusted. Note that the proximal anchor 494 and/or distal anchor 492 could be provided with such locks to permit the proximal anchor 494 and/or distal anchor 492 to be slidingly moved along the bridge 496. The use of such locks on the proximal anchor 494 and/or distal anchor 492 could be in addition to, or in lieu of, the use of the lock 522 on the middle anchor 500.

Bridge structures similar to those of FIGS. 22 and 23, as well as other bridge structure embodiments that can be used as an entire bridge and/or one or more bridge portions with the current invention, are described in pending U.S. patent application Ser. No. 11/144,521, entitled “Devices and Methods for Percutaneous Repair of the Mitral Valve via the Coronary Sinus” and filed on Jun. 3, 2005, the entire contents of which are incorporated herein.

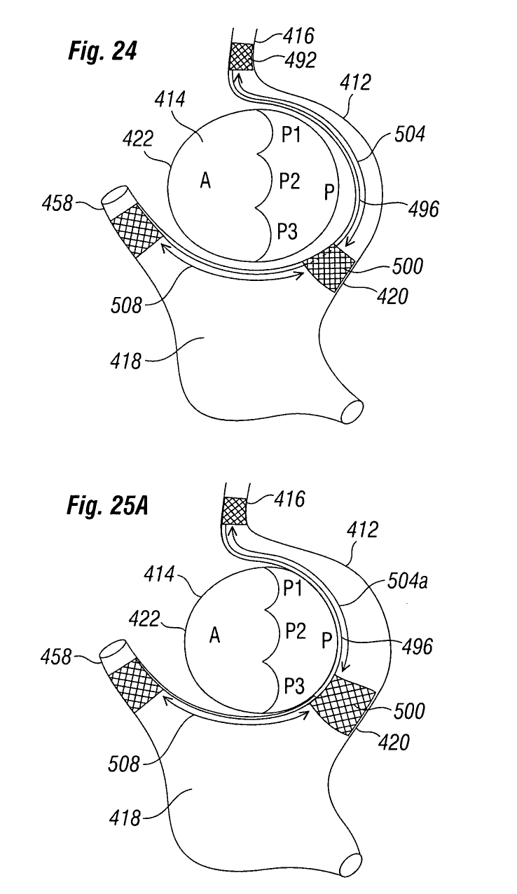

FIG. 24 depicts a three-anchor implant 490 deployed to reshape a mitral valve 414. The distal anchor 492 is deployed in a distal portion 416 of the coronary sinus 412, the middle anchor 500 is deployed adjacent the ostium 420, and the proximal anchor 494 is deployed in the superior vena cava 458. The mitral valve annulus 422 is thus almost completely encircled by the implant 490.

The implant 490 can be deployed using various methods, including the general methods depicted and described previously with respect to FIGS. 19A-20D, but with the additional step of deploying the middle anchor 500, and (depending on the particular embodiment) possibly adjusting the distal bridge length and/or proximal bridge length. In one embodiment, the distal anchor is deployed first, followed by the middle anchor, and then the proximal anchor. Other orders of anchor deployment are also within the scope of the invention.

As was the case with the two-anchor implant of the invention, the distal anchor 492 and proximal anchor 494 of the three-anchor implant 490 according to the invention can be deployed at various locations. In one embodiment, the distal anchor 492 is deployed beyond the P1 commissure or between the P1 and P2 commissures; the middle anchor 500 could be deployed just inside or outside of the coronary ostium; and the proximal anchor 494 could be deployed in the superior vena cava or inferior vena cava, or within the atrium. Note that other deployment locations for the anchors are also within the scope of the invention, with the particular deployment location dependent on various factors such as the particular application. For example, in treating a mitral valve 412, the distal anchor 492 could be deployed anywhere within the coronary sinus 412, and the proximal anchor 494 could be deployed anywhere from the ostium 420, right atrium 418, superior vena cava 458, or inferior vena cava 456. Depending on the particular application, the middle anchor 500 could be deployed anywhere between the deployed locations of the distal anchor 492 and proximal anchor 494.

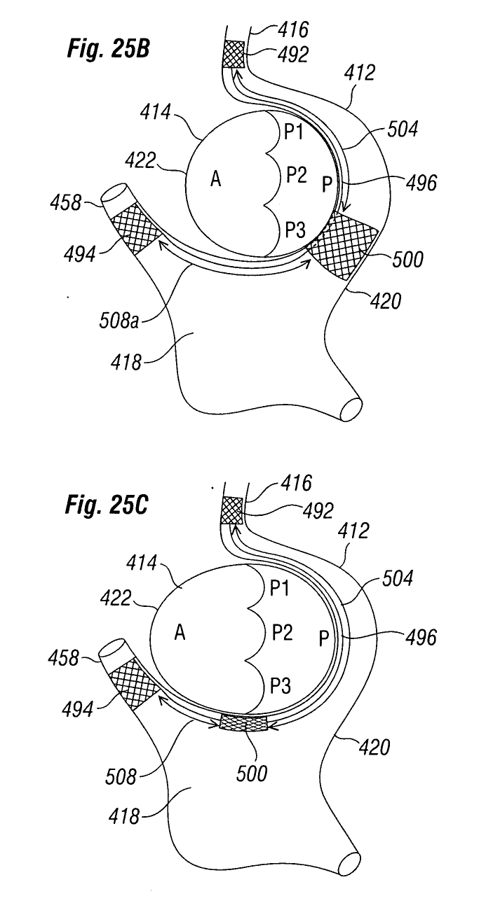

FIG. 25A depicts a three-anchor implant 490 with the anchors deployed in the same positions as in FIG. 24, but having a distal bridge length 504a shorter than that depicted in FIG. 24, thereby increasing the adjustment of adjacent leaflet P, and particularly of cusps P1 and P2.

FIG. 25B depicts a three-anchor implant 490 with the anchors deployed in the same positions as in FIGS. 24 and 25A, but having a shorter proximal bridge length 508a, thereby providing increased tension adjacent leaflet A.

FIG. 25C depicts a three-anchor implant 490, with the middle anchor 500 intentionally positioned adjacent the junction of leaflets A, P, thus permitting a user to selectively vary tension at leaflet A (by adjusting the proximal bridge length 508) or vary tension on leaflet P (by adjusting distal bridge length 504). The user could thus deploy the respective anchors as desired (including deploying the middle anchor 500 adjacent the anterior/posterior leaflet junction), and then adjust the proximal bridge length 508 and/or distal bridge length 504 while monitoring leaflet coaptation to achieve the desired repositioning of the anterior leaflet A and/or posterior leaflet P.

Depending on the particular embodiment, after the proximal, middle, and distal anchors are deployed, the separation distance between the anchors created by the bridge and bridge portions may be adjusted. The particular approach to adjusting the separation distance depends on the particular implant embodiment and application. Adjusting of the separation distance may be performed by the user and/or by inherent characteristics of the implant.

Once the anchors are deployed, the proper placement of the implant is confirmed, and (where applicable) the lengths of the respective bridge portions are properly adjusted, the delivery catheter can be removed from the patient's body with the implant remaining inside the patient. The efficacy of the implant and its deployed position can be confirmed and monitored at various times during and after the deployment procedure via various techniques, including visualization methods such as fluoroscopy.

Various materials could be used to form the implant, delivery catheter, and other system components. For example, the inner member and/or outer sheath could be formed of braided or non-braided polymeric components. The fluoroscopic marker bands could comprise gold or other relatively highly radiopaque materials.

While the invention has been described with reference to particular embodiments, it will be understood that various changes and additional variations may be made and equivalents may be substituted for elements thereof without departing from the scope of the invention or the inventive concept thereof. For example, it will be recognized that the embodiments described above and aspects thereof may also be used to treat a triscuspid valve or other valves in substantially similar manner. In addition, many modifications may be made to adapt a particular situation or device to the teachings of the invention without departing from the essential scope thereof. Therefore, it is intended that the invention not be limited to the particular embodiments disclosed herein, but that the invention will include all embodiments falling within the scope of the appended claims.

What is claimed is:

1. An implant for placement in a heart of a patient, comprising:
a first anchor configured to be secured to tissue in a coronary sinus;
a second anchor configured to be secured to tissue in a right atrium; and
a bridge extending from the first anchor to the second anchor.

2. The implant of claim 1, wherein the first anchor comprises a radially expandable and generally cylindrical structure.

3. The implant of claim 1, wherein the second anchor comprises one or more barbs configured to engage atrial tissue

4. The implant of claim 1, wherein the second anchor comprises an expandable mesh-like structure.

5. The implant of claim 1, wherein the first anchor is configured to be deployed in a distal portion of the coronary sinus, and the bridge has sufficient length to extend from the distal portion of the coronary sinus to the right atrium.

6. The implant of claim 1, wherein a length along the bridge between the first anchor and second anchor is 40 mm to 150 mm.

7. The implant of claim 6, wherein one or more of the anchors is configured to be advanced along a portion of the bridge.

8. An implant for placement in a heart of a patient, comprising:
a first anchor configured to be secured to cardial tissue;
a second anchor configured to be secured to tissue in a vena cava; and
a bridge extending from the first anchor to the second anchor.

9. The implant of claim 8, wherein the first anchor is configured to be secured to tissue in a coronary sinus.

10. The implant of claim 9, wherein the first anchor comprises a radially expandable generally cylindrical structure.

11. The implant of claim 9, wherein the bridge has sufficient length to extend from a distal portion of the coronary sinus to an inferior vena cava.

12. The implant of claim 9, wherein the bridge has sufficient length to extend from the distal portion of the coronary sinus to a superior vena cava.

13. The implant of claim 9, wherein the second anchor is configured to be secured to tissue in an inferior vena cava, and the first anchor is configured to be secured to tissue in a superior vena cava.

14. A method of repairing a mitral valve, comprising:
obtaining an implant having a first anchor, a second anchor, and a bridge connecting the first anchor to the second anchor;
deploying the first anchor in a first portion of the heart; and
deploying the second anchor in a second portion of the heart such that the bridge engages against an atrial septum with sufficient force to reshape an annulus of the mitral valve.

15. The method of claim 14, wherein the second portion of the heart is in the right atrium.

16. The method of claim 14, wherein the second portion of the heart is an inferior vena cava.

17. The method of claim 14, wherein the second portion of the heart is a superior vena cava.

18. The method of claim 17, wherein the first portion of the heart is an inferior vena cava.

19. The method of claim 14, wherein the first portion of the heart is a coronary sinus.

20. An implant for placement in a heart of a patient, comprising:
a distal anchor configured to be secured to tissue in a coronary sinus;
a middle anchor configured to be secured to heart tissue;
proximal anchor configured to be secured to heart tissue outside of the coronary sinus;
a distal bridge portion extending from the distal anchor to the middle anchor; and
a proximal bridge portion extending from the middle anchor to the proximal anchor.

21. The implant of claim 20, wherein the distal anchor is configured to be secured to tissue in a distal portion of the coronary sinus, and the middle anchor is configured to be secured to tissue at or adjacent the coronary ostium.

22. The implant of claim 20, wherein the middle anchor is configured to be secured to tissue at or adjacent the coronary ostium, and the proximal anchor is configured to be secured to tissue in a vena cava.

24. The implant of claim 20, wherein the distal bridge portion and proximal bridge portion form a generally continuous bridge structure.

25. The implant of claim 24, wherein the middle anchor is configured to be slidingly moved along the generally continuous bridge structure.

26. The implant of claim 20, wherein the proximal anchor is configured to be slidingly moved along the proximal bridge portion.

27. A method of treating a mitral valve in a patient's heart, comprising:
obtaining an implant having a distal anchor, middle anchor, and proximal anchor, with a distal anchor portion extending from the distal anchor to the middle anchor, and a proximal anchor portion extending from the middle anchor to the proximal anchor;
deploying the distal anchor in a coronary sinus;
deploying the middle anchor in the heart; and
deploying the proximal anchor in the heart.

28. The method of claim 27, wherein deploying the middle anchor comprises deploying the middle anchor at or adjacent the coronary sinus ostium.

29. The method of claim 28, wherein deploying the distal anchor comprises deploying the distal anchor in a distal portion of the coronary sinus.

30. The method of claim 27, wherein deploying the proximal anchor comprises deploying the proximal anchor in the right atrium.

31. The method of claim 27, wherein deploying the proximal anchor comprises deploying the proximal anchor in a superior vena cava.

32. The method of claim 27, wherein deploying the proximal anchor comprises deploying the proximal anchor in an inferior vena cava.

33. The method of claim 27, wherein the proximal bridge portion and distal bridge portion connect to form a generally continuous bridge structure, and the method further comprising:
slidingly moving the middle anchor along the bridge structure.

34. The method of claim 27, further comprising:
slidingly moving the proximal anchor along the proximal bridge portion.

35. The method of claim 27, wherein the distal bridge portion defines a length between the distal anchor and middle anchor, and the method further comprising:
adjusting the length between the distal anchor and middle anchor.

36. The method of claim 27, wherein the proximal bridge portion defines a length between the proximal anchor and middle anchor, and the method further comprising:
adjusting the length between the proximal anchor and middle anchor.
